(19) United States
(12) Patent Application Publication
(10) Pub. No.: US 2013/0177973 A1
(43) Pub. Date: Jul. 11, 2013

(54) Flow Cytometer

(71) Applicant: Yasushi KONDO, Kyoto-shi (JP)

(72) Inventor: Yasushi KONDO, Kyoto-shi (JP)

(73) Assignee: SHIMADZU CORPORATION, Kyoto-shi (JP)

(21) Appl. No.: 13/536,632

(22) Filed: Jun. 28, 2012

(30) Foreign Application Priority Data
Jul. 1, 2011 (JP) 2011-147108

(51) Int. Cl. C12Q 1/04

(52) U.S. Cl. CPC C12Q 1/04
USPC 435/288.7

(57) ABSTRACT

A flow cytometer for a microanalysis and a fast detection is provided. In the flow cytometer, the downstream portion of a flow cell is fitted with an optical detection system including a laser emitter and a light detector, an imaging system including a high-speed camera and a stroboscopic lamp, and a cell sorter. The high-speed camera and the stroboscopic lamp illuminates and photographs candidate particles based on a photographing trigger signal provided by a trigger generator. The trigger signal is provided by the trigger generator when a predetermined time has elapsed from the moment when an examination particle determined to be a candidate particle has been measured by the optical detection system. The high-speed camera photographs multiple images for a given period of time from the moment when the trigger signal is provided, and sends the image data to a target particle detection unit of a data processor.

### TECHNICAL FIELD

The present invention relates to a flow cytometer capable of a microanalysis and a fast detection.

### BACKGROUND ART

Flow cytometers are apparatuses in which particles are made to flow in a channel where a laminar flow is formed, and in which each particle is sequentially analyzed. They are often used in the fields of molecular biology and medicine, particularly in order to analyze cells. Flow cytometers are also used to selectively collect target particles from among those which have been analyzed.

“Cytometry” on the website of Beckman Coulter, Inc. (Non-Patent Document 1) describes an apparatus for delivering a laser light to each particle, detecting multiple kinds of lights, such as scattered light and fluorescence generated by irradiation, and statistically analyzing the detection data of these lights to sort the particles. “Flow Cytometry ImageStream of BM Equipment Co., Ltd.” on the website of BM Equipment Co. Ltd (Non-Patent Document 2) introduces a flow cytometer for photographing each particle and sorting the particles based on the morphological difference of each particle analyzed from the image.

### BACKGROUND ART DOCUMENT

### Patent Document

[Patent Document 1] WO-A1 2009/031301

### Non-Patent Document

[Non-Patent Document 1]: “Cytometry,” [online], Beckman Coulter, Inc., Internet, [Jun. 17, 2011]
[Non-Patent Document 2]: “BM Equipment Co., Ltd. Flow Cytometry ImageStream,” [online], BM Equipment Co., Ltd., Internet, [Jun. 17, 2011]
[Non-Patent Document 3]: K. Goda, K. K. Tsia & B. Jalali, “Serial time-encoded amplified imaging for real-time observation of fast dynamic phenomena,” Nature (U.K.), 30 Apr. 2009, vol. 458, pp. 1145-1149

### DISCLOSURE OF THE INVENTION

### Problem to be Solved by the Invention

In the medical field, for example, it is required to detect the presence or non-presence of cancer stem cells in collected blood, for the early detection of cancer. However, even if they are present, the number of cancer stem cells in the blood will be extremely small: only a few cells in a billion will be detected.

With the apparatus of Non-Patent Document 1, as many as tens to hundreds of thousands of cells are measured per second and sorted according to their optical characteristics. However, it is difficult to specify cancer stem cells alone, and therefore the sorted cells also include numerous other cells which have similar optical properties to cancer stem cells. One method to separate the cancer stern cells from the other cells is as follows: magnetic materials are attached to proteins which are easily combined with cancer stem cells, and the proteins are mixed with a group of selectively collected cells. Then, the cancer stem cells which have been combined with the proteins are collected with a magnet. However, this method is expensive and requires extra work and time.

Although the apparatus of Non-Patent Document 2 is capable of specifying only cancer stem cells by analyzing the images to obtain the morphology, the measuring speed is disadvantageously slow. For example, Non-Patent Document 2 states that cells can be analyzed at the rate of a thousand cells per second. Checking a billion cells at this rate takes as many as about twelve days. Hence, an acceleration of the analysis is required in some way. However, speeding up the analysis of the apparatus of Non-Patent Document 2 means that a large number of images must be taken, which causes problems such as the amount of data to be handled is too large and takes too much time to process.

The problem to be solved by the present invention is to provide a flow cytometer capable of a microanalysis and a fast detection.

### Means for Solving the Problem

To solve the aforementioned problem, the present invention provides a flow cytometer for detecting target particles having a predetermined morphology from among examination particles, including:

a flow path through which the examination particles flow;

a flow rate controller for controlling the flow rate of the examination particles flowing through the flow path;

a light emitter for emitting light onto a predetermined detection area in the flow path;

a light detector for detecting light from the detection area;

a candidate particle determiner for determining whether or not the examination particles flowing through the detection area are candidate particles having optical properties of the target particles based on the output from the light detector;

a photographing unit for taking an image of a predetermined photographing area which is downstream of the detection area in the flow path;

a photographing timing instructor for instructing the photographing unit of timings for taking the image of the candidate particles flowing through the photographing area, based on a flow path length between the detection area and the photographing area as well as the flow rate; and

a target particle detector for detecting the target particles from the candidate particles based on the morphology of the candidate particles by examining the images taken by the photographing unit.

The flow cytometer according to the present invention may further include:

a selective collector for selectively collecting the target particles at a predetermined selective collection area which is downstream of the photographing area in the flow path; and

a selective collection timing instructor for instructing the selective collector of the timings for selectively collecting the target particles based on the flow path length between the photographing area and the selective collection area as well as the flow rate.

In the apparatus of Non-Patent Document 2, images are taken and analyzed continuously. Conversely, the flow cytometer according to the present invention takes the following two steps: the optical properties of examination particles are obtained by the light emitter and the light detector, and then the particles are narrowed down to candidate particles by the candidate particle determiner and their images are taken. Images are thereby taken and analyzed intermittently in the flow cytometer of the present invention. When detecting a small amount of particles, such as cancer stem cells, detection of the target particles does not take place in short, consecutive intervals. Accordingly, the amount of image data is decreased, allowing enough time to process the data. This enables a higher flow rate.

If the target particles are to be detected from among a billion examination particles inside a few minutes, for example, a detection rate of several million particles per second is required. Accordingly, the photographing unit must perform at a great speed, such as several million frames per second. Typical photographing units used for such fast photographing include a burst charge coupled device (CCD) image sensor or a burst complementary metal oxide semiconductor (CMOS) image sensor, as described in Patent Document 1. Burst CCD image sensors and CMOS image sensors aim to increase the photographing speed by using signals provided from each pixel that are memorized for a predetermined number of frames in memory units arranged around a pixel area, which are then collectively read out. Although the apparatus of Patent-Document 1 is not suitable for continuous photographing, it is highly suitable for intermittent photographing, as in the present invention, thanks to its fast photographing speed.

### Effects of the Invention

In the flow cytometer according to the present invention, images are taken and analyzed intermittently at appropriate timings, which decreases the amount of image data and speeds up the data processing. This allows the flow rate in the flow path to be increased as well, enabling a small amount of particles to be detected in a shorter time than ever before.

### BRIEF DESCRIPTION OF THE DRAWINGS

FIG. 1 is a schematic configuration diagram of the flow cytometer according to the present invention.

FIG. 2 is a diagram for explaining the method of determining the candidate particles by using a histogram.

FIGS. 3A through 3C show diagrams for explaining the method of determining the candidate particles by using shapes of peaks.

FIG. 4 is a schematic configuration diagram of the optical detection system in the case where a serial time-encoded amplified microscopy is used.

### BEST MODES FOR CARRYING OUT THE INVENTION

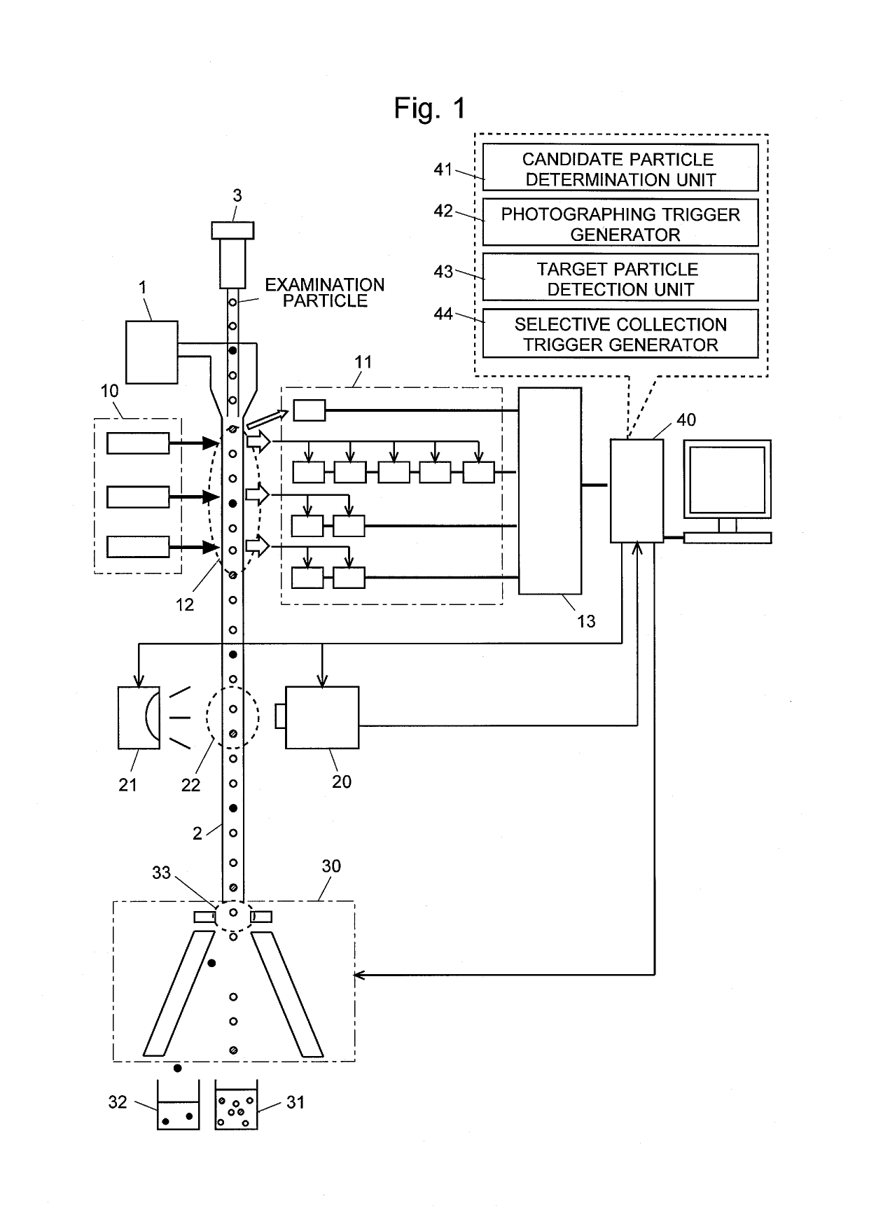

One embodiment of the flow cytometer according to the present invention will be described, with reference to the attached figures. FIG. 1 is a schematic configuration diagram of the flow cytometer of the present embodiment.

In the flow cytometer of FIG. 1, a sheath fluid is introduced from a flow controller 1, and the flow of the sheath fluid is controlled so that a laminar flow at a constant flow rate is formed in a flow cell 2. Examination particles introduced, from a sample introduction unit 3 to the flow cell 2 flow in order through the flow cell 2 in which the laminar flow is formed.

Starting from upstream, the flow cell 2 is fitted with, in order, an optical detection system that includes a laser emitter 10 and a light detector 11, an imaging system that includes a high-speed camera 20 and a stroboscopic lamp 21, and a cell sorter 30.

The laser emitter 10 includes one or more laser light sources, and emits a laser light onto a detection area 12 of the flow cell 2. The light detector 11 includes one or more light detection units arranged around the detection area 12, and detects transmitted light, reflected light, scattered light, fluorescence, and other types of light which result from the laser light irradiating the examination particles flowing through the detection area 12.

Each detection unit of the light detector 11 sends a detection signal to the signal processor 13 corresponding to the intensity of received light. The detection signal is subjected to a predetermined signal processing, such as amplification and digitization, and is then sent to a data processor 40.

The data processor 40 is formed of, for example, a personal computer in which a predetermined data processing program has been installed. The data processor 40 includes, as function blocks, a candidate particle determination unit 41, a photographing trigger generator 42, a target particle detection unit 43, and a selective collection trigger generator 44.

Based on the input data from the signal processor 13, the candidate particle determination unit 41 determines whether or not the optical properties of the examination particles correspond to those of the target particles. This determination by the candidate particle determination unit 41 can be performed using one of various statistical methods, such as a histogram method, a gating method, and other methods described in Non-Patent Document 1, for example.

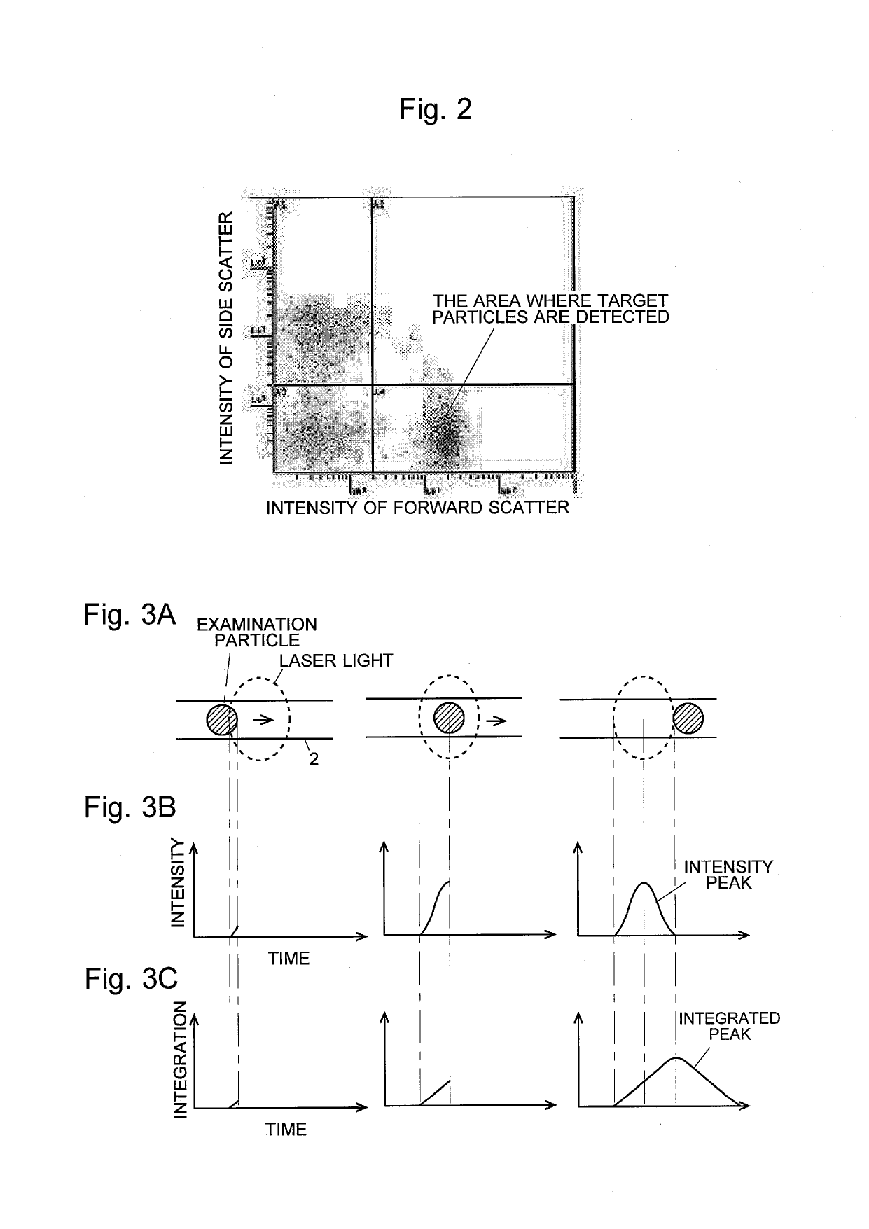

A determination method using a histogram will now be described in brief. Using the detection data obtained from the detection units of the light detector 11 for the parameters, plotting each of the examination particles on the graph provides a frequency distribution graph as shown in FIG. 2. This frequency distribution graph is called a histogram. FIG. 2 illustrates a two-parameter histogram showing the correlation between the intensity of side scatter and that of forward scatter.

By means of a preliminary experiment, creating a histogram (as shown in FIG. 2) in advance will display the area of the histogram in which the target particles will appear. Therefore, the appearance of examination particle detection data in a predetermined area on the histogram makes it easy to determine whether or not that particle is a candidate particle for the target particle.

If an examination particle being analyzed is determined not to be a candidate particle by the candidate particle determination unit 41, it passes through the imaging system without being photographed. Examination particles which have passed through the imaging system are not selectively collected by the cell sorter 30, and drop into the container 31.

If an examination particle being analyzed is determined to be a candidate particle by the candidate particle determination unit 41, a detailed measurement is performed by the imaging system. Which is downstream of the optical detection system. A specific measurement procedure of the optical detection system will now be described.

The imaging system includes the high-speed camera 20 and the stroboscopic lamp 21, and illuminates as well as photographs candidate particles upon receiving photographing trigger signal from the photographing trigger generator 42. After an examination particle determined to be a candidate particle by the candidate particle determination unit 41 has been measured by the optical detection system, a predetermined delay time elapses, and at this point in time the photographing trigger signal is provided from the photographing trigger generator 42. This delay time is determined by the flow path length between the detection area 12 and the photographing area 22 of the flow cell 2 as well as the flow rate set by the flow control 1.

The high-speed camera 20 sequentially takes multiple images for a given period of time from the point when the photographing trigger signal is provided. Multiple images are taken of each candidate particle so that an appropriate image can be selected from among them in case the candidate particle was rotating while passing through the photographing area.

The data of the images taken by the high-speed camera 20 are sent to the target particle detection unit 43 in the data processor 40. The target particle detection unit 43 performs an image processing, such as binarization, on each piece of the image data, and then computes the degree of similarity the image data of the target particle which has been photographed in advance. If a piece of image data is found that has a degree of similarity equal to or larger than a predetermined threshold, the photographed candidate particle is determined to be a target particle.

If a photographed candidate particle is determined to be a target particle by the target particle detection unit 43, the selective collection trigger generator 44 provides a selective collection trigger signal to the cell sorter 30 at the point when a predetermined delay time has elapsed from the moment the photos were taken by the imaging system. Accordingly, a target particle which arrives at the selective collection area 33 of the cell sorter 30 is selectively collected into the container 32. This delay time is determined by the length of the flow path between the photographing area and the selective collection area 33 of the flow cell 2, as well as the flow rate set by the flow controller 1.

The selective collection by the cell sorter 30 may be performed, for example, by the method described in Non-Patent Document 1: a droplet containing a target particle which drops from the outlet end of the flow cell 2 is electrically charged, and a particular electric field is applied to the droplet while it is falling down in order to alter the direction of the fall toward the container 32.

If a photographed candidate particle is determined not to be a target particle by the target particle detection unit 43, an electric field is not applied, to the droplet containing the candidate particle in the cell sorter 30. The droplet will just fall straight into the container 31.

Thus far, the processing of each unit of the flow cytometer according to the present embodiment has been schematically described. The way in which a faster measurement is achieved by using this configuration will now be described.

Generally speaking, the time required for data processing by the target particle detection unit 43 is the factor that prevents the faster measurement of the flow cytometer. Hence, the data to be processed may exceed the processing capacity due to a flow rate that is high, the photographing of all examination particles by the imaging system, and the data processing of the images by the target particle detection unit 43. In the apparatus of the present embodiment, however, data processing is intermittently performed only on candidate particles in the target particle detection unit 43. Therefore, if candidate particles appear at a sufficiently low rate, the target particle detection unit 43 can perform data processing without delay, even if the flow rate is high.

Specifically, if data processing can be performed at a thousand per second by the target particle detection unit 43, and if the probability of the appearance of a candidate particle is one in one hundred particles at most, the apparatus of the present embodiment can perform a fast measurement of a million particles per second.

A fast measurement as just described requires the photographing speed of the high-speed camera 20 to be at least the same as the measurable number (measuring speed) of particles per unit time (a hundred thousand frames per second). In practice, it is desirable to obtain multiple images for one particle. Hence, in the case where ten images are taken for one particle, the required photographing speed is a million frames per second. For a camera capable of such fast photographing, a burst CCD image sensor or a burst CMOS image sensor described in Patent Document 1 can be used.

The detection speed and accuracy of refinement in the optical detection system and the candidate particle determination unit 41 must be able to deal with the measuring speed of the apparatus. The higher the refinement accuracy is, the lower the probability of the appearance of a candidate particle becomes, enabling a faster measurement speed.

Therefore, it is important to select the appropriate method of detection and determination in the optical detection system and the candidate particle determination unit 41 in accordance with the optical properties of the target particle. The following method can be used for detection and determination, in addition to the statistical methods described in the aforementioned embodiment.

FIGS. 3A through 3C show the temporal change in intensity of light transmitted when an examination particle passes through an area where a laser is being emitted (FIG. 3A). As shown in FIG. 3B, the intensity of received light is detected as a peak in each light detection unit. By passing the detection signal to an integrator, an integrated peak as shown in FIG. 3C is obtained. The shapes of these peaks convey the information according to the shape and characteristics of the examination particle. Therefore, it is possible to determine whether or not the target particle is a candidate particle by comparing the shapes of the examination particle's intensity peak and integrated peak of the examination particle with those of the target particle.

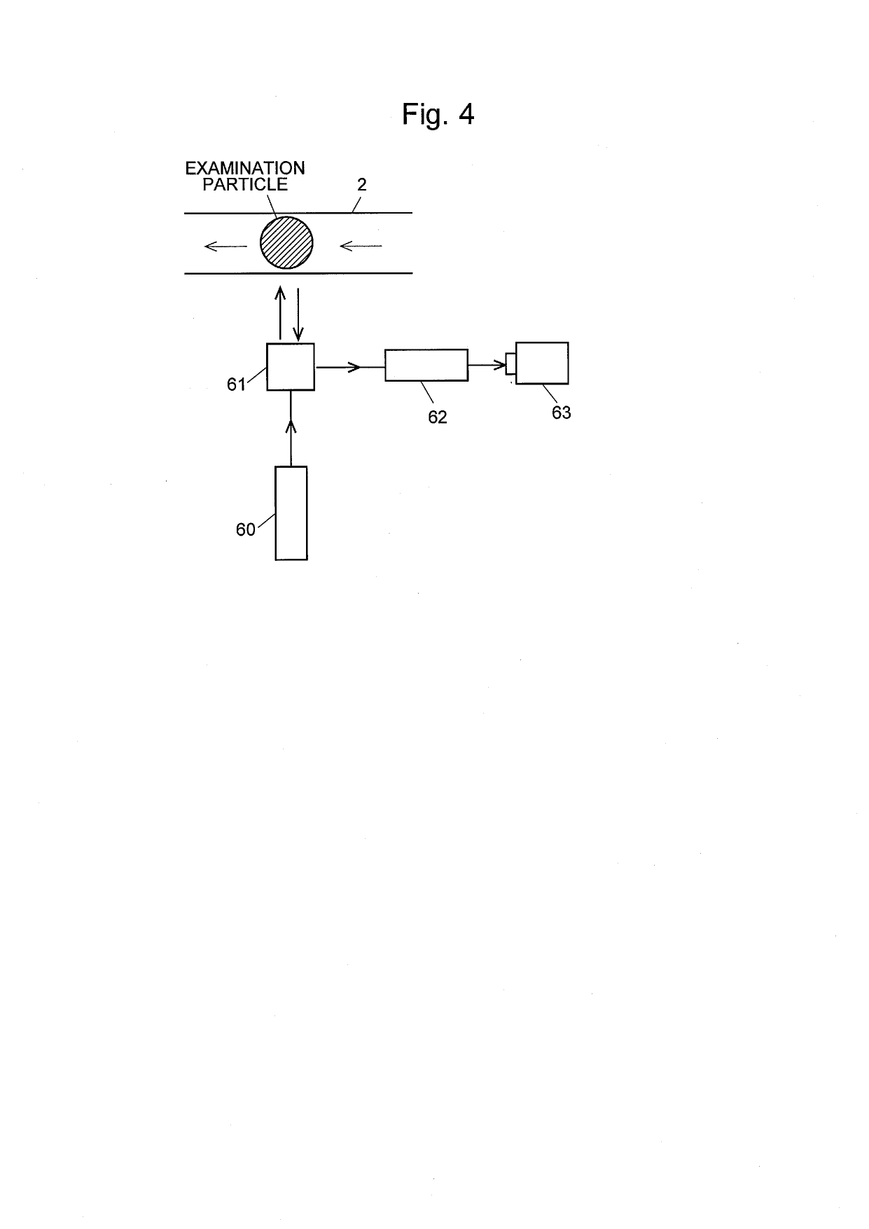

The detection and determination can also be performed by using the principle of serial time-encoded amplified microscopy (STEAM), which is described in Non-Patent Document 3 (K. Goda, K. K. Tsia & B. Jalali. “Serial time-encoded amplified imaging for real-time observation of fast dynamic phenomena,” Nature (U.K.), 30 Apr. 2009, vol. 458, pp. 1145-1149). The principle of STEAM will now be described with reference to FIG. 4.

In STEAM, a laser pulse with a sufficiently wide range of wavelength is emitted from a laser emitter 60 at fixed time intervals, and each pulse is dispersed two-dimensionally according to the wavelength by a two-dimensional dispersion unit 61. The laser light of each wavelength dispersed by the two-dimensional dispersion unit 61 is delivered to and reflected by respective position on the sample. The reflected laser light of each wavelength passes backward through the two-dimensional dispersion unit 61 to return to one pulse. This pulse passes through a Fourier transformer 62, where the pulse is transformed from the frequency domain to the time domain, and then detected by a photodiode 63.

An example of the graph of the intensity of received light detected by the photodiode 63 is shown in FIG. 2d of Non-Patent Document 3, in serial time-encoded amplified microscopy, the frequency (wavelength) corresponds to the position on a sample, and the frequency components are converted into time-domain components by the Fourier transformer 62. Therefore, the time-domain component has the information on the position on the sample. That is, in the graph, the two-dimensional intensity distribution is converted to a time series. Carrying out a Fourier inverse transform on the temporal change of the intensity signal of each pulse obtained in this way can provide the surface structure information for the examination particle. In the case where serial time-encoded amplified microscopy is applied to the flow cytometer of the present embodiment, comparing the pattern of temporal intensity change of each pulse with that of the target particle (which was obtained in advance by an actual measurement or computation) makes it possible to determine whether or not a particle is a candidate particle without having to perform a Fourier inverse transform.

Thus far, the flow cytometer according to the present invention has been described by using the embodiments. It is evident that it can be changed within the spirit of the present invention.

### EXPLANATION OF NUMERALS

1 . . . Flow Controller
2 . . . Flow Cell
3 . . . Sample Introduction Unit
10 . . . Laser Emitter
11 . . . Light Detector
12 . . . Detection Area
13 . . . Signal Processor
20 . . . High-Speed Camera
21 . . . Stroboscopic Lamp
22 . . . Photographing Area
30 . . . Cell Sorter
31, 32 . . . Container
33 . . . Selective Collection Area
40 . . . Data Processor
41 . . . Candidate Particle Determination Unit
42 . . . Photographing Trigger Generator
43 . . . Target Particle Detection Unit
44 . . . Selective Collection Trigger Generator
60 . . . Laser Emitter
61 . . . Two-Dimensional Dispersion Unit
62 . . . Fourier Transformer
63 . . . Photodiode

1. A flow cytometer for detecting target particles having a predetermined morphology from among examination particles, comprising:
a flow path through which the examination particles flow;
a flow rate controller for controlling a flow rate of the examination particles flowing through the flow path;
a light emitter for emitting light onto a predetermined detection area in the flow path;
a light detector for detecting light from the detection area;
a candidate particle determiner for determining whether or not the examination particles flowing through the detection area are candidate particles having optical properties of the target particles based on an output from the light detector;
a photographing unit for taking an image of a predetermined photographing area which is downstream of the detection area in the flow path;
a photographing timing instructor for instructing the photographing unit of timings for taking the image of the candidate particles flowing through the photographing area, based on a flow path length between the detection area and the photographing area as well as the flow rate; and
a target particle detector for detecting the target particles from the candidate particles based on a morphology of the candidate particles by examining the images taken by the photographing unit.

2. The flow cytometer according to claim 1, further comprising:
a selective collector for selectively collecting the target particles at a predetermined selective collection area which is downstream of the photographing area in the flow path; and
a selective collection timing instructor for instructing the selective collector of timings for selectively collecting the target particles based on a flow path length between the photographing area and the selective collection area as well as the flow rate.

3. The flow cytometer according to claim 1, wherein the photographing unit is either a burst CCD image sensor or a burst CMOS image sensor which comprises a plurality of memory units each corresponding to each pixel of a pixel area and collectively reads out images of a predetermined number of frames.

4. The flow cytometer according to claim 1, wherein the candidate particle determiner determines the candidate particles by using a statistical method.

5. The flow cytometer according to claim 1, wherein the candidate particle determiner determines the candidate particles based on a shape of a temporal change peak of an intensity of received light and a shape of an integrated peak of the intensity of received light.

6. The flow cytometer according to claim 1, wherein the candidate particle determiner determines the candidate particles based on a pattern of a temporal intensity change obtained by a serial time-encoded amplified microscopy.

7. The flow cytometer according to claim 2, wherein the photographing unit is either a burst CCD image sensor or a burst CMOS image sensor which comprises a plurality of memory units each corresponding to each pixel of a pixel area and collectively reads out images of a predetermined number of frames.

8. The flow cytometer according to claim 2, wherein the candidate particle determiner determines the candidate particles by using a statistical method.

9. The flow cytometer according to claim 3, wherein the candidate particle determiner determines the candidate particles by using a statistical method.

10. The flow cytometer according to claim 7, wherein the candidate particle determiner determines the candidate particles by using a statistical method.

11. The flow cytometer according to claim 2, wherein the candidate particle determiner determines the candidate particles based on a shape of a temporal change peak of an intensity of received light and a shape of an integrated peak of the intensity of received light.

12. The flow cytometer according to claim 3 wherein the candidate particle determiner determines the candidate particles based on a shape of a temporal change peak of an intensity of received light and a shape of an integrated peak of the intensity of received light.

13. The flow cytometer according to claim 7 wherein the candidate particle determiner determines the candidate particles based on a shape of a temporal change peak of an intensity of received light and a shape of an integrated peak of the intensity of received light.

14. The flow cytometer according to claim 2, wherein the candidate particle determiner determines the candidate particles based on a pattern of a temporal intensity change obtained by a serial time-encoded amplified microscopy.

15. The flow cytometer according to claim 3, wherein the candidate particle determiner determines the candidate particles based on a pattern of a temporal intensity change obtained by a serial time-encoded amplified microscopy.

16. The flow cytometer according to claim 7, wherein the candidate particle determiner determines the candidate particles based on a pattern of a temporal intensity change obtained by a serial time-encoded amplified microscopy.
